(19) United States
(12) Patent Application Publication
(10) Pub. No.: US 2010/0120921 A1
(43) Pub. Date: May 13, 2010

(54) RENIN INHIBITORS FOR TREATMENT OF HYPERTENSION IN PATIENTS WITH HIGH SODIUM DIET

(75) Inventor: Alan N. Charney, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/528,187

(22) PCT Filed: Feb. 28, 2008
(86) PCT No.: PCT/US2008/055207

§ 371 Date: Aug. 21, 2009

Related U.S. Application Data
(60) Provisional application No. 60/892,389, filed on Mar. 1, 2007.

(51) Int. Cl. A61K 31/165; A61P 9/12

(52) U.S. Cl. 514/620

(57) ABSTRACT

The present invention relates to methods for the prevention of, delay of progression to, or treatment of hypertension in a patient with a high sodium diet, comprising administering to such a patient a therapeutically effective amount of a renin inhibitor or a pharmaceutically acceptable salt thereof.

The present invention relates to therapeutic methods involving the administration of renin inhibitors, such as aliskiren, or a pharmaceutically acceptable salt thereof. In particular, the present invention provides advantageous methods for treating hypertension comprising in particular aliskiren, preferably, a hemi-fumarate salt thereof.

### INTRODUCTION

In the following the term “aliskiren”, if not defined specifically, is to be understood both as the free base and as a salt thereof, especially a pharmaceutically acceptable salt thereof, most preferably a hemi-fumarate thereof.

Renin released from the kidneys cleaves angiotensinogen in the circulation to form the decapeptide angiotensin I. This is in turn cleaved by angiotensin converting enzyme in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure and other complications of hypertension such as stroke.

The renin inhibitor, aliskiren, in particular, a hemi-fumarate thereof, is known to be effective as a treatment for reducing blood pressure irrespective of age, sex or race and is also well tolerated. Aliskiren in form of the free base is represented by the following formula

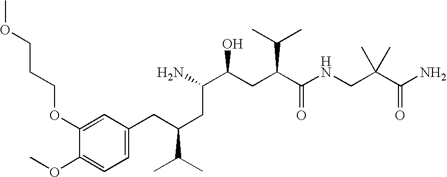

and chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. As described above, most preferred is the hemi-fumarate salt thereof which is specifically disclosed in EP 678503 A as Example 83.

The blood pressure of many people is affected by dietary sodium, and dietary sodium has been recognized as an independent factor leading to an increased blood pressure. Despite the fact that life style modifications are recommended for all hypertensive patients, specifically the recommendation that patients ingest low sodium diets, patients with hypertension continue to require drug therapy to control their blood pressure. Even with drug therapy the blood pressures of almost 40% of hypertensive patients are not controlled. One reason for this lack of control could be the relatively high salt diet ingested by most people in the West. Thus, previous antihypertensive therapy has not always been efficient to address the negative effects of a high sodium diet with respect to the blood pressure increase in that such a diet may counteract the beneficial blood pressure lowering effects observed with an antihypertensive agent.

While the exact mechanism how sodium works to to elevate blood pressure is not completely understood, it is believed that too much salt causes the sodium channels (structures that move sodium into and out of cells) to work too hard and gradually the channels begin to fail.

Given the reality that not all patients are ready to make the necessary changes to their lifestyle by cutting down the sodium intake in their diet, it is important to provide an effective and safe therapy to treat hypertension in patients that have a high sodium diet and to avoid an increase of blood pressure due to a high sodium diet in patients treated for hypertension.

### SUMMARY OF THE INVENTION

After intense investigations it was found surprisingly that renin inhibitors such as aliskiren, unlike many other antihypertensive agents, reduce blood pressure efficiently irrespective of the sodium intake of the patient to be treated, thus providing a safe and efficient therapeutic method for treating hypertension in these patients.

The present invention is therefore related to a method for the prevention of, delay progression to or treatment of hypertension in a patient with a high sodium diet, comprising administering to such a patient a therapeutically effective amount of a renin inhibitor or a pharmaceutically acceptable salt thereof.

The present invention also relates to a method for the prevention of, delay progression to or treatment of the effects of a high sodium diet on blood pressure, comprising administering to a patient in need thereof a therapeutically effective amount of a renin inhibitor or a pharmaceutically acceptable salt thereof.

Thus, with the present invention the blood pressure is controlled more effectively in patients with a high sodium diet. This is a marked benefit observed with renin inhibitors.

### DETAILED DESCRIPTION OF THE INVENTION

Listed below are some of the definitions of various additional terms used herein to describe certain aspects of the present invention. However, the definitions used herein are those generally known in the art, e.g., hypertension, and apply to the terms as they are used throughout the specification unless they are otherwise limited in specific instances.

The term “prevention” refers to prophylactic administration to healthy patients to prevent the development of the conditions mentioned herein. Moreover, the term “prevention” means prophylactic administration to patients being in a pre-stage of the conditions to be treated. This is also referred to a primary prevention. In addition the term “prevention” encompasses also “secondary prevention,” which refers to the administration to patients who already have had a condition in order to prevent its recurrence or worsening, or to prevent the complications that may arise from the condition.

The term “delay the onset of”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “hypertension” refers to a condition where the pressure of blood within the blood vessels is higher than normal as it circulates through the body. When the systolic pressure exceeds 140 mmHg or the diastolic pressure exceeds 90 mmHg for a sustained period of time, damage is done to the body. Populations at increased risk due to other conditions, such as diabetes, are recommended to have even lower levels than cited above. Excessive systolic pressure can rupture blood vessels, and when it occurs within the brain, a stroke results. Hypertension may also cause thickening and narrowing of the blood vessels which ultimately could lead to atherosclerosis. The term “hypertension” as used herein is meant to encompass various types of hypertension, such as those described hereinafter, namely severe hypertension, pulmonary hypertension, malignant hypertension, and isolated systolic hypertension.

The term “severe hypertension” refers to hypertension characterized by a systolic blood pressure of ≧180 mmHg and a diastolic blood pressure of ≧110 mmHg.

The term “pulmonary hypertension” (PH) refers to a blood vessel disorder of the lung in which the pressure in the pulmonary artery rises above normal level of ≦25/10 (especially primary and secondary PH), e.g., because the small vessels that supply blood to the lungs constrict or tighten up. According to the WHO, PH may be divided into five categories: pulmonary arterial hypertension (PAH), a PH occurring in the absence of a known cause is referred to as primary pulmonary hypertension, while secondary PH is caused by a condition selected, e.g., from emphysema; bronchitis; collagen vascular diseases, such as scleroderma, Crest syndrome or systemic lupus erythematosus (SLE); PH associated with disorders of the respiratory system; PH due to chronic thrombotic or embolic disease; PH due to disorders directly affecting the pulmonary blood vessels; and pulmonary venous hypertension (PVH).

The term “malignant hypertension” is usually defined as very high blood pressure with swelling of the optic nerve behind the eye, called papilledema (grade IV Keith-Wagner hypertensive retinopathy). This also includes malignant HTN of childhood.

The term “isolated systolic hypertension” refers to hypertension characterized by a systolic blood pressure of ≧140 mmHg and a diastolic blood pressure of <90 mmHg.

The term “renovascular hypertension” (renal artery stenosis) refers to a condition where the narrowing of the renal artery is significant which leads to an increase of the blood pressure resulting from renin secretion by the kidneys. Biomarkers include renin, PRA and prorenin.

The term “antihypertensive effect” refers to a control of the blood pressure to normal. Preferably, normal blood pressure is characterized by a goal blood pressure of <140 mmHg, preferably <138 mmHg, systolic pressure and <90 mmHg diastolic pressure. In preferred embodiments, the antihypertensive effect refers to a mean sitting diastolic blood pressure of below 89 mm Hg, preferably below 88 mmHg, more preferably 87 mmHg or below. In other preferred embodiments, the antihypertensive effect refers to a mean sitting systolic blood pressure of below 140 mmHg, preferably 139 mmHg, more preferably 138 mmHg or below.

The term “baseline level” refers to the blood pressure level of the treated subject prior to the therapy with the renin inhibitor to treat hypertension. The baseline level refers to either or both the systolic and the diastolic blood pressure. Consequently, the baseline level for systolic pressure can be ≧140 mmHg, such as ≧150 mmHg, or ≧160 mmHg, depending on the individual, and the baseline level for the diastolic pressure can be ≧90 mmHg, such as ≧95 mmHg, depending on the individual.

The term “high sodium diet” refers to high levels of dietary sodium based on the dietary sodium in hypertension literature, as well as advice from cardiovascular clinicians and cardiovascular dietitians. Typically, it refers to a dietary sodium intake of ≧200 mmol/day, such as 200 to 320 mmol/day, in particular 220 to 300 mmol/day.

The term “low sodium diet” refers to lower levels of dietary sodium based on the dietary sodium in hypertension literature, as well as advice from cardiovascular clinicians and cardiovascular dietitians. Typically, it refers to a dietary sodium intake of 50 to 150 mmol/day, such as 80 to 100 mmol/day in particular ≦100 mmol/day.

The term “equivalent” in the context of “the blood pressure in said patient can be controlled to be equivalent to the blood pressure of a patient with a low sodium diet undergoing the same treatment” refers to a non-inferiority in the antihypertensive effect with a high sodium diet in contrast to a low sodium diet. Typically the high sodium diet group will be considered to be statistically non-inferior to the low sodium diet group in the change in 24-hour mean ambulatory systolic blood pressure if the upper limit of the confidence interval for μ_{HS}-μ_{LS}, where μ_{HS} and μ_{LS} are the mean in changes from baseline in 24-hour ambulatory systolic blood pressure for the patients groups with the high and low sodium diet, respectively, does not exceed 4 mmHg (EMEA, CPMP/EWP/482/99. Points to consider on switching between superiority and non-inferiority. London, July 2000).

Suitable renin inhibitors include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-prolyl-L-phenylalanyl-N-[2-hydroxy-5-methyl-1-(2-methylpropyl)-4-[[[2-methyl-1-[[(2-pyridinylmethyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexyl(methyl)-2-hydroxy-3-(1-methylethoxy)-3-oxopropyl]-S-methyl-L-cysteinamide); and zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), preferably, in each case, the hydrochloride salt thereof, SPP630, SPP635 and SPP800 as developed by Speedel.

Preferred renin inhibitor of the present invention include RO 66-1132 and RO 66-1168 of formulae (I) and (II)

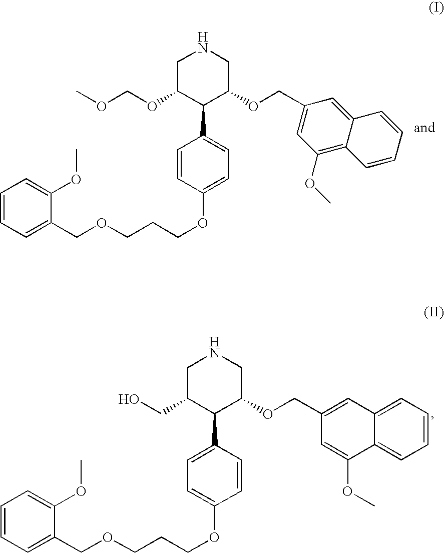

respectively, or a pharmaceutically acceptable salt thereof.

In particular, the present invention relates to a renin inhibitor which is a δ-amino-γ-hydroxy-ω-aryl-alkanoic acid amide derivative of the formula

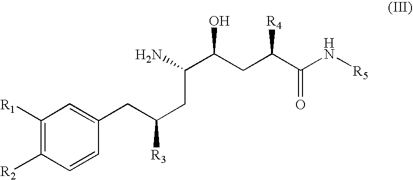

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆-aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (III) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyryloxyethyl.

As a C₁₋₆aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxy-carbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are δ-amino-γ-hydroxy-ω-aryl-alkanoic acid amide derivatives of formula (III) having the formula

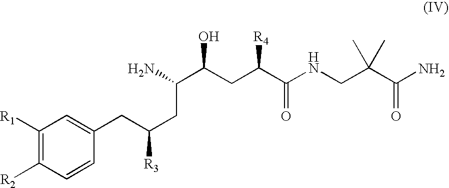

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren and as represented by formula (V).

The term “aliskiren”, if not defined specifically, is to be understood both as the free base and as a salt thereof, especially a pharmaceutically acceptable salt thereof, most preferably a hemi-fumarate salt thereof.

The renin inhibitor of formula (V) is preferably in the form of a hemi-fumarate salt.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo. The corresponding active ingredients or pharmaceutically acceptable salts thereof may also be used in form of a solvate, such as a hydrate or including other solvents, used for crystallization.

The compounds can be present as pharmaceutically acceptable salts. If these compounds have, for example, at least one basic center, they can form acid addition salts. Corresponding acid addition salts can also be formed having, if desired, an additionally present basic center. Compounds having an acid group (for example COOH) can also form salts with bases.

The compounds may be present in prodrug form. The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:

Oxidative Activation
N- and O-dealkylation
Oxidative deamination
N-oxidation
Epoxidation

Reductive Activation
Azo reduction
Sulfoxide reduction
Disulfide reduction
Bioreductive Alkylation
Nitro reduction.

Also to be mentioned as metabolic activations of prodrugs are nucleotide activation, phosphorylation activation and decarboxylation activation. For additional information, see “The Organic Chemistry of Drug Design and Drug Action”, R B Silverman (particularly Chapter 8, pages 497 to 546), incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, although protected derivatives of compounds of the invention may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the invention.

The pharmaceutical preparations described herein may be for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubalizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical preparation according to the present invention are therapeutically effective dosages, especially those which are commerically available.

Normally, in the case of oral administration, an approximate daily dose of from about 1 mg to about 2 g is to be estimated e.g. for a patient of approximately 75 kg in weight.

The dosage of the active compound can depend on a variety of factors, such as mode of administration, homeothermic species, age and/or individual condition.

The pharmaceutical preparation will usually be supplied in the form of suitable dosage unit form, for example, a capsule or tablet, and comprising an appropriate amount of a combination as disclosed herein.

A solid oral dosage form comprises a capsule or more preferably a tablet or a film-coated tablet.

A solid oral dosage form according to the invention comprises additives or excipients that are suitable for the preparation of the solid oral dosage form according to the present invention. Tabletting aids, commonly used in tablet formulation can be used and reference is made to the extensive literature on the subject, see in particular Fiedler's “Lexicon der Hilfstoffe”, 4th Edition, ECV Aulendorf 1996, which is incorporated herein by reference. These include, but are not limited to, fillers, binders, disintegrants, lubricants, glidants, stabilising agents, fillers or diluents, surfactants, film-formers, softeners, pigments and the like.

In a preferred embodiment the solid oral dosage form according to the present invention comprises as an additive a filler.

In a preferred embodiment the solid oral dosage form according to the present invention comprises as an additive, in addition to a filler, a disintegrant.

In a preferred embodiment the solid oral dosage form according to the present invention comprises as an additive, in addition to a filler and a disintegrant, a lubricant.

In a preferred embodiment the solid oral dosage form according to the present invention comprises as an additive, in addition to a filler, a disintegrant and a lubricant, a glidant.

In a preferred embodiment the solid oral dosage form according to the present invention comprises as an additive, in addition to a filler, a disintegrant, a lubricant and a glidant, a binder.

As fillers one can particularly mention starches, e.g., potato starch, wheat starch, corn starch, hydroxypropyl cellulose, hydroxyethyl cellulose, hydroxypropyl methyl cellulose (HPMC) and, preferably, microcrystalline cellulose, e.g., products available under the registered trade marks AVICEL, FILTRAK, HEWETEN or PHARMACEL.

As binders for wet granulation, one can particularly mention polyvinylpyrrolidones (PVP), e.g., PVP K 30, HPMC, e.g., viscosity grades 3 or 6 cps, and polyethylene glycols (PEG), e.g., PEG 4000. A most preferred binder is PVP K 30.

As disintegrants one can particularly mention carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum, most preferably crosslinked PVP (CROSPOVIDONE), crosslinked CMC (Ac-Di-Sol), carboxymethylstarch-Na (PIRIMOJEL and EXPLOTAB). A most preferred disintegrant is CROSPOVIDONE.

As glidants one can mention in particular colloidal silica, such as colloidal silicon dioxide, e.g., AEROSIL, magnesium (Mg) trisilicate, powdered cellulose, starch, talc and tribasic calcium phosphate or combinations of these with fillers or binders, e.g., silicified microcrystalline cellulose (PROSOLV). A most preferred glidant is colloidal silicon dioxide (e.g. AEROSIL 200).

As fillers or diluents one can mention confectioner's sugar, compressible sugar, dextrates, dextrin, dextrose, lactose, mannitol, microcrystalline cellulose, in particular, having a density of about 0.45 g/cm³, e.g., AVICEL, powdered cellulose, sorbitol, sucrose and talc. A most preferred filler is microcrystalline cellulose.

As lubricants one can mention in particular Mg stearate, aluminum (Al) or Ca stearate, PEG 4000 to 8000 and talc, hydrogenated castor oil, stearic acid and salts thereof, glycerol esters, Na-stearylfumarate, hydrogenated cotton seed oil and others. A most preferred lubricant is Mg stearate.

Additives to be used as filmcoating materials comprise polymers such as HPMC, PEG, PVP, polyvinylpyrrolidone-vinyl acetate copolymer (PVP-VA), polyvinyl alcohol (PVA), and sugar as film formers. A most preferred coating material is HPMC, especially HPMC 3 cps (preferred amount 5-6 mg/cm²), and mixtures thereof with further additives, e.g., those available under the registered trade mark OPADRY. Further additives comprise pigments, dies, lakes, most preferred TiO₂ and iron oxides, anti-tacking agents like talk and softeners like PEG 3350, 4000, 6000, 8000 or others. Most preferred additives are talk and PEG 4000.

The doses of renin inhibitor such as one of formula (V) to be administered to warm-blooded animals, for example human beings, of, for example, approximately 70 kg body weight, especially the doses effective in the inhibition of the enzyme renin, e.g. in lowering blood pressure may be from approximately 3 mg to approximately 3 g, particularly from approximately 10 mg to approximately 1 g, for example approximately from 20 mg to 600 mg (e.g. 150 mg to 300 mg), per person per day. Single doses comprise, for example, 75, 100, 150, 200, 250, 300 or 600 mg per adult patient. Usually, children receive about half of the adult dose or they can receive the same dose as adults. The dose necessary for each individual can be monitored and adjusted to an optimum level. The usual recommended starting dose of a renin inhibitor of formula (V) is usually 150 mg once daily. In some patients whose blood pressure is not adequately controlled, the daily dose may be increased to 300 mg. The renin inhibitor of formula (V) may be used over a dosage range of 150 mg to 300 mg administered once daily. All doses are based on the active agent, i.e. the free base.

Ultimately, the exact dose of the active agent and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

### EXAMPLE 1

Composition of aliskiren 150 mg (free base) uncoated tablets in mg/unit.

| | Roller | | | |
|---|---|---|---|---|
| | compacted | Dosage | Dosage | Dosage |
| Component | tablet | form 1 | form 2 | form 3 |
| Aliskiren hemi-fumarate | 165.750 | 165.750 | 165.750 | 165.750 |
| Microcrystalline cellulose | 220.650 | 84.750 | 72.250 | 107.250 |
| Polyvinylpyrrolidon K 30 | — | — | 12.000 | 12.000 |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

Composition of aliskiren 150 mg (free base) uncoated tablets in % by weight.

| | Roller | | | |
|---|---|---|---|---|
| | compacted | Dosage | Dosage | Dosage |
| Component | tablet | form 1 | form 2 | form 3 |
| Aliskiren hemi-fumarate | 34.53 | 55.25 | 55.25 | 48.75 |
| Microcrystalline cellulose | 45.97 | 28.25 | 24.08 | 31.545 |
| Polyvinylpyrrolidon K 30 | — | — | 4 | 3.53 |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of aliskiren 150 mg (free base) uncoated tablets in mg/unit (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren hemi-fumarate | 165.75 | 165.75 | 165.75 | 165.75 |
| Phase | Microcrystalline | 220.65 | 84.75 | 72.25 | 90.25 |
| | cellulose | | | | |
| | Polyvinylpyrrolidon K 30 | — | — | 12.00 | 12.00 |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline | — | — | — | 17.00 |
| | cellulose | | | | |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of aliskiren 150 mg (free base) uncoated tablets in % by weight (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren hemi-fumarate | 34.53 | 55.25 | 55.25 | 48.75 |
| Phase | Microcrystalline | 45.97 | 28.25 | 24.08 | 26.545 |
| | cellulose | | | | |
| | Polyvinylpyrrolidon K 30 | — | — | 4 | 3.530 |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline | — | — | — | 5 |
| | cellulose | | | | |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### EXAMPLE 2

Composition of aliskiren (dosage form 3) film-coated tablets in mg/unit.

| | Dosage form 3/Strength | | |
|---|---|---|---|
| | 75 mg | 150 mg | |
| Component | (free base) | (free base) | 300 mg (free base) |
| Aliskiren hemi-fumarate | 82.875 | 165.750 | 331.500 |
| Microcrystalline cellulose | 53.625 | 107.250 | 214.500 |
| Polyvinylpyrrolidon K 30 | 6.000 | 12.000 | 24.000 |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated tablet | 180.000 | 357.000 | 706.000 |
| weight | | | |

### EXAMPLE 3

### Clinical Studies

The effect of Aliskiren (300 mg) on reduction in mean 24-hour ambulatory blood pressure systolic hypertensive patients using low and high sodium diets is examined in a 12-week, randomized, open-label, multi-center, cross-over study. Subjects with systolic hypertension have a 2-4 week washout of their current antihypertensive medications. Patients who meet 8-hour ABPM criteria at week 0 (ABPM mean daytime systolic blood pressure ≧135 mmHg and <160 mmHg) are randomized to either a high sodium (≧200 mmol/day) or low sodium (≦100 mmol/day) diet and will be dispensed aliskiren 300 mg. The daytime 8 hr ABPM criterion of > or =135/85 for entry into the trial is based on data suggesting this level corresponds to cuff blood pressure measurements of > or =140/90, and that the risks and complications of hypertension increase above this level (Pickering 2005, Verdicchia 2000).

After 2 and 4 weeks on the assigned sodium level diet, 24-hour urine (Bentley, Brook 2006) collections for sodium and creatinine are collected and measured to confirm adherence to the diet. After 4 weeks on the assigned sodium level diet, 24-hour ABPM is measured. Patients are then crossed over to the other level sodium diet. Those patients who had high sodium diet for the first four weeks start having low sodium diet for the next 4 weeks and vice versa. After 2 and 4 weeks on the new diet, 24-hour urine collections for sodium and creatinine are again collected and measured to confirm adherence to the diet. After 4 weeks on the newly assigned sodium level diet, 24-hour ABPM are measured. If at any time the office mean sitting systolic blood pressure (MSSBP) is ≧180 and/or the office mean sitting diastolic blood pressure is ≧110, the patient should be discontinued from the study and put back on their antihypertensive medication(s). As a result of this study, it was found that the antihypertensive effect of aliskiren is the same (within the BP boundaries indicated) regardless of whether the subject ingests a high or a low sodium diet.

In detail the study involves the following:

TABLE 1
Study outline
Population

| Phase | Pre-randomization | Randomized Diet Order | | | | |
|---|---|---|---|---|---|---|
| Period | Screening/ | Initial four-weeks | | | Final four- | |
| | Washout | of diet | | | weeks of diet | |
| Visit | 1^{1,2} | 2, 3^{2,3} | 4 | 5, 6³ | 7 | 8, 9³ |
| Duration | 2-4 week(s) | 4 weeks | | | 4 weeks | |
| Day | −28 or −14 | 0 | 14 | 28 | 42 | 56 |
| Randomization | | X | | | | |

| | | |
|---|---|---|
| Rx | None | Open Label aliskiren 300 mg |

¹If the patient is required to be tapered off the current antihypertensive medication then the tapering should occur at Visit 1. The patient should be washed out of their antihypertensive medication for at least 2 weeks prior to Visit 2.
²For currently untreated patients Visit 1 and Visit 2 will be combined into one visit.
³There is one day between visits 2 & 3, 5 & 6 and 8 & 9. Randomization will occur at Visit 3.

The study population consists of 122 male or female patients with systolic hypertension (ABPM mean daytime systolic blood pressure ≧135 mmHg and <160 mmHg) from approximately 20 centers in the US.

### Inclusion/Exclusion Criteria

The investigator must ensure that all patients who meet the following inclusion and exclusion criteria are offered enrollment in the study. No additional exclusions can be applied by the investigator, in order that the study population will be representative of all eligible patients.

### Inclusion Criteria:

1. Male or female outpatients 18-60 years old
2. Subjects with systolic hypertension who meet 24 hour ABPM criteria (ABPM mean daytime systolic blood pressure ≧135 mmHg and <160 mmHg)

### Exclusion Criteria:

1. Renal artery stenosis or other causes of secondary hypertension.
2. Overt heart failure or a history of heart failure within the preceding six months.
3. Second or third degree heart block.
4. History of MI or CVA within the preceding six months.
5. Unstable angina pectoris.
6. Clinically relevant arrhythmias, i.e. any arrhythmia being symptomatic or requiring medical therapy.
7. Clinically significant valvular heart disease
8. Type 1 or Type 2 diabetes mellitus or abnormal HbA1c
9. MDRD estimated GFR <60 ml/min/1.73 m²
10. BMI>30
11. Subjects taking more than 2 antihypertensive medications.
12. Use of other investigational drugs at the time of enrollment within 30 days
13. Flomax® (tamsulosin hydrochloride) and other alpha blockers.
14. Antiarrhythmic drugs, including digoxin.
15. History of hypersensitivity to any of the study drugs or to drugs with similar chemical structures
16. History of malignancy of any organ system, treated or untreated, within the past 5 years whether or not there is evidence of local recurrence or metastases, with the exception of localized basal cell carcinoma of the skin
17. Pregnant or nursing (lactating) women, where pregnancy is defined as the state of a female after conception and until the termination of gestation, confirmed by a positive hCG laboratory test (>5 mlU/ml)
18. Women of child-bearing potential (WOCBP), defined as all women physiologically capable of becoming pregnant, including women whose career, lifestyle, or sexual orientation precludes intercourse with a male partner and women whose partners have been sterilized by vasectomy or other means, UNLESS they meet the following definition of post-menopausal: 12 months of natural (spontaneous) amenorrhea or 6 months of spontaneous amenorrhea with serum FSH levels >40 mlU/m or 6 weeks post surgical bilateral oophorectomy with or without hysterectomy OR are using one or more of the following acceptable methods of contraception: surgical sterilization (e.g., bilateral tubal ligation), hormonal contraception (implantable, patch, oral), and double-barrier methods. Reliable contraception should be maintained throughout the study and for 7 days after study drug discontinuation.
19. Fertile males, defined as all males physiologically capable of conceiving offspring UNLESS the patient and his partner agree to comply with acceptable contraception, including the items listed under exclusion criteria number 18.

### Premature Patient Withdrawal

Patients must be withdrawn from the study for any of the following reasons:
Withdrawal of informed consent
Pregnancy
Study drug discontinuation

Patients also should be withdrawn at any time if the investigator concludes that it would be in the patient's best interest for any reason. Protocol violations should not lead to patient withdrawal unless they indicate a significant risk to the patient's safety.

Patients may voluntarily withdraw from the study for any reason at any time. They may be considered withdrawn if they state an intention to withdraw, or fail to return for visits, or become lost to follow up for any other reason.

If premature withdrawal occurs for any reason, the investigator must determine the primary reason for a patient's premature withdrawal from the study and record this information on the Study Completion CRF.

For patients who are lost to follow-up (i.e., those patients whose status is unclear because they fail to appear for study visits without stating an intention to withdraw), the investigator should show “due diligence” by documenting in the source documents steps taken to contact the patient, e.g., dates of telephone calls, registered letters, etc.

Patients who are prematurely withdrawn from the study will not be replaced by an equal number of newly enrolled patients.

### Treatment

### Investigational and Control Drugs

Open-label aliskiren 300 mg is packaged in bottles of 35 tablets.

### Treatment Arms

Patients are randomized to two initial diet orders (high sodium diet and low sodium diet) at Visit 3, Week 0+1 day. After 4 weeks diet orders will be crossed over. That is, diet for those patients who were taking high sodium diet for the first four weeks from baseline are changed to low sodium diet at Visit 6, Week 4+1 day and vice versa.

Thus, patients are assigned to one of the following two (2) arms in a 1:1 ratio at randomization (Visit 3, Week 0+1 day):

Arm1: Patients with high sodium diet for the first four weeks and with low sodium diet for the next four weeks;
Arm2: Patients with low sodium diet for the first four weeks and with high sodium diet for the next four weeks.

### Treatment Blinding

This is an open label, blinded endpoint study.

Patients, investigator staff, persons performing the assessments, and data analysts are unblinded to both treatment information and sodium diet information post randomization. They remain blinded to 24 hour ABPM results during the study until database lock.

### Treating the Patient

### Instructions for Prescribing and Taking the Study Drug

Patients are instructed to take one tablet every morning between 8-10 AM except on the morning of their study visit when they will be dosed in the office.

### Permitted Study Drug Dose Adjustments and Interruptions

Study drug dose adjustments and/or interruptions are not permitted.

### Rescue Medication

Use of rescue medication is not permitted.

### Other Concomitant Treatment

Use of the following concomitant medications may interfere with the evaluation of safety, tolerability and/or efficacy. Therefore, these medications are excluded throughout the trial from the beginning of Visit 1 until the end of the study (Visit 9). Patients who are receiving such medication(s) are excluded, or if ethically justified, the medication(s) may be withdrawn according to the manufacturer's/investigator's instructions prior to Visit 2:
1. Drugs approved for the treatment of hypertension even if prescribed for another indication.
2. Diuretics of any kind
3. Potassium supplements will not be allowed at study entry. Use of potassium supplements will be allowed for management of hypokalemia during the randomized study drug treatment phase.
4. Flomax® (tamsulosin hydrochloride) and other alpha blockers.
5. Antidepressant drugs in the class of MAO inhibitors. Other psychotropic drugs will be allowed if well tolerated when previously taken and if the dosage is stable 3 months prior to Visit 1 and expected to remain constant throughout the study.
6. Oral corticosteroids.
7. Thyroid medication and/or estrogen replacement therapy, unless these have been stable maintenance replacement doses for 3 months preceding Visit 1 or Visit 2.
8. Chronic administration (defined as administration >3 days per week) of sympathomimetic drugs such as those found in nasal decongestants (e.g., pseudoephedrine, phenylpropanolamine) and bronchodilators (e.g., metaproterenol) is not allowed. Acute use of these drugs (if not within 48 hours of a scheduled visit) is allowed.
9. Ergot and serotonin (5-hydroxytryptamine) receptor agonists (e.g., sumatriptan).
10. Phosphodiesterase type-5 (PDE-5) inhibitors such as sildenafil (Viagra®), vardenafil (Levitra®), and tadalafil (Clalis®) are not allowed within 48 hours prior to any scheduled visit.
11. Antiarrhythmic drugs, including digoxin.

### Study Drug Discontinuation

Patients with a MSDBP ≧110 mmHg and/or a MSSBP ≧180 mmHg at any time during the study must be permanently discontinued from study drug. Appropriate antihypertensive therapy should be initiated in the above patients at the discretion of the investigator.

Patients with signs or symptoms of hypotension and/or MSDBP <60 mmHg and/or a MSSBP <100 mmHg at any time during the study, (i.e., washout or post randomization) should be thoroughly evaluated by the investigator and if clinically warranted, must be permanently discontinued from study drug.

Patients who become pregnant during the study must be permanently discontinued from study drug.

Patients who discontinue study drug after randomization and before completing the study should be scheduled for a visit as soon as possible, at which time all of the assessments listed at Visit 8 are performed.

At a minimum, all patients who discontinue study drug, including those who refuse to return for a final visit, are contacted for safety evaluations during the 30 days following the last dose of study drug.

Patients who discontinue study drug should be considered withdrawn from the study after the final visit assessments are performed or when it is clear that the patient will not return for these assessments.

Patients who use any of the prohibited medications should be discontinued.

In addition to these requirements for study drug discontinuation, the investigator should discontinue study drug for a given patient if, on balance, he thinks that continuation would be detrimental to the patient's well-being.

### Visit Schedule and Assessments

Table 2 lists all of the assessments and indicates with an “X” the visits when they are performed.

Patients should be seen for all visits on the designated day or as close to it as possible.

Patients who discontinue study drug before completing the study, and those who prematurely withdraw from the study for any reason, should be scheduled for a visit as soon as possible, at which time all of the assessments listed for Visit 8 are performed.

Patients who discontinue study drug also should return for the assessments indicated by an asterisk (*) in Table 2. If the patient refuses to return for these assessments or are unable to do so, every effort should be made to contact them or a knowledgeable informant by telephone to determine the status of the patient.

At a minimum, the patient is contacted for safety evaluations during the 30 days following the last dose of study drug, including final contact at the 30-day point. Documentation of attempts to contact the patient should be recorded in the patient record.

TABLE 2
Assessment Schedule

| | | Screening | Baseline | | Treatment Period | | | | | |
|---|---|---|---|---|---|---|---|---|---|---|
| | | Visit number | | | | | | | | |
| | | ¹1 | ²2 | 3 | 4 | ²5 | 6 | 7 | ²8 | 9 |
| | | Day | | | | | | | | |
| | | −28 | 1 | 2 | 14 | 28 | 29 | 42 | 56 | 57 |
| | | Week | | | | | | | | |
| | Category | −4 | 0 | 0 + 1 day | 2 | 4 | 4 + 1 day | 6 | 8 | 8 + 1 day |
| Inclusion/Exclusion | S | X | X | | | | | | | |
| criteria | | | | | | | | | | |
| Information & | S | X | | | | | | | | |
| Informed consent | | | | | | | | | | |
| Discontinue/Taper | S | X | | | | | | | | |
| Antihypertensive | | | | | | | | | | |
| Meds | | | | | | | | | | |
| Medical History | D | X | | | | | | | | |
| Physical examination | S | X | | | | X | | | ⁷X* | |
| Prior/Concomitant | D | X | X | X | X | X | X | X | ⁷X* | X |
| Medication | | | | | | | | | | |
| Dispense study | S | | | X | | | X | | | |
| medication | | | | | | | | | | |
| ⁵Dosage | D | | | X | X | | X | X | | X |
| Administration | | | | | | | | | | |
| Record | | | | | | | | | | |
| Adverse events | D | | X | X | X | X | X | X | ⁷X* | X |
| Vital Signs | D | X | X | | X | X | | X | ⁷X* | |
| 24 hour ABPM | D | | X | | | X | | | X | |
| 24 hour ABPM | D | | | X | | | X | | | X |
| removal | | | | | | | | | | |
| Serum creatinine | D | | X | | | X | | | ⁷X* | |
| Serum HbA1c | D | X | | | | | | | | |
| 24-hour urinary | D | | X | | X | X | | X | X | |
| sodium and | | | | | | | | | | |
| creatinine | | | | | | | | | | |
| PRA, Plasma | D | | X | | | X | | | X* | |
| Angiotensin II, | | | | | | | | | | |
| Plasma Aldosterone | | | | | | | | | | |
| ³Hematology and | D | X | | | | | | | ⁷X* | |
| blood chemistry | | | | | | | | | | |
| Urinalysis | D | X | | | | | | | ⁷X* | |
| ⁴Pregnancy Test | D | X | X | | | | | | ⁷X* | |
| Dietary | S | X | | | | | | | | |
| Questionnaire | | | | | | | | | | |
| Study Completion | D | | | | | | | | | X* |
| Comments | D | X | X | X | X | X | X | X | X | X |
| Screening Log | D | X | | | | | | | | |

¹Patients who have been newly diagnosed with uncomplicated hypertension and who have not taken any anti-hypertensive medications for 4 weeks prior to Visit 1 may combine Visits 1 and 2 and can be enrolled directly into the treatment period. If Visits 1 and 2 are combined, all assessments planned for both visits must be completed.
²ABPM device will be applied on the first day of Visit 2 to qualified patients and data will be down loaded on the next day. ABPM may be repeated once within the 48 hours of the completion of the first attempt should it fail to meet the quality control. Randomization will begin after successful completion of the mean 8-hr ABPM and having a mean 8-hr daytime ABPM SBP ≧135 mmHg and <160 mmHg. Details about ABPM procedure is provided in
³Lipid profile (total cholesterol, triglycerides, HDL-C, LDL-C, VLDL-C) will be completed at Visits 1 & 8. Electrolytes, blood urea and creatinine are included in the complete laboratory assessments.
⁴Women of childbearing potential only. A serum pregnancy test is to be performed at Visits 1 & 8 and a local urine pregnancy test at Visit 2.
⁵At Visits 4 and 7 discuss compliance with patient and confirm sufficient supply is remaining for next visit. At Visits 5 and 8 collect, count, and record in the Drug Accountability Log the number of unused tablets.
⁷These assessments should also be done for patients who discontinue study drug

### Visit 1 (Week-4: Day-28, Screening)

Patients should be seen for all visits between 7:00 a.m. and 10:00 a.m. The following procedures are performed:
Obtain written informed consent for the study; informed consent must be obtained prior to performing any procedures related to the trial and after the patient has received sufficient information about the study, the opportunity to ask any questions and consider the options.
Document in the patient's medical records that (s)he is participating in this clinical trial (CSPP100AUS02), informed consent has been obtained and a copy of the signed consent has been given to the patient.
Assessment of inclusion and exclusion criteria.
A complete medical history/demography
Prior and concomitant medications are recorded. Prior medications taken during the 30 day period before screening (Study Visit 1) are documented.
Blood pressure and pulse rate measurements are performed in the sitting and standing positions. Three blood pressure readings and one pulse rate will be taken in the sitting position. After the sitting measurements, one standing blood pressure and pulse rate are taken.
Complete physical examination is performed.
Blood and urine samples is collected in the fasting state for routine safety laboratory evaluations. A serum pregnancy test is performed for women of child bearing potential only.
Serum HbA1c measured in the fasting date.

### Visit 2 (Week 0)

Study Visit 2 should take place 2-4 weeks +/−3 days after Study Visit 1. Patients should be seen for all visits between 7:00 A.M. and 10:00 A.M.

The following procedures are performed:
Reassessment of inclusion/exclusion criteria.
Blood pressure and pulse rate measurements are performed in the sitting and standing positions. Three blood pressure readings and one pulse rate are taken in the sitting position. After the sitting measurements, one standing blood pressure and pulse rate is taken.
Assessment of any concomitant medications taken or changed since the last visit.
Assessment of any adverse events that occurred since the previous study visit.
Apply 24-hour ABPM
Collect blood for serum creatinine, PRA, plasma aldosterone, plasma angiotensin II
Collect 24-hour urine for urinary sodium and creatinine
Perform urine pregnancy in office
   Visit 3—Randomization (Week 0+1 day)

Study Visit 3 should take place 1 day after Study Visit 2. Patients should be seen for all visits between 7:00 A.M. and 10:00 A.M.

The following procedures are performed:
Assessment of any concomitant medications taken or changed since the last visit.
Assessment of any adverse events that occurred since the previous study visit.
24-hour ABPM is removed
Diet packet given to patient
Assign sodium level diet

### Visit 4 (Week 2: Day 14)

Study Visit 4 should take place 2 weeks +/−3 days after Study Visit 3. Patients should be seen for all visits between 7:00 A.M. and 10:00 A.M.

The following procedures are performed:
Blood pressure and pulse rate measurements are performed in the sitting and standing positions. Three blood pressure readings and one pulse rate are taken in the sitting position. After the sitting measurements, one standing blood pressure and pulse rate is taken.
Assessment of any concomitant medications taken or changed since the last visit.
Assessment of any adverse events that occurred since the previous study visit.
Collect 24-hour urine for urinary sodium and creatinine

### Visit 5 (Week 4: Day 28)

Study Visit 5 should take place 2 weeks +/−3 days after Study Visit 4. Patients should be seen for all visits between 7:00 A.M. and 10:00 A.M.

The following procedures are performed:
Blood pressure and pulse rate measurements are performed in the sitting and standing positions. Three blood pressure readings and one pulse rate are taken in the sitting position. After the sitting measurements, one standing blood pressure and pulse rate is taken.
A complete physical exam is performed.
Assessment of any concomitant medications taken or changed since the last visit.
Assessment of any adverse events that occurred since the previous study visit.
Apply 24-hour ABPM
Collect blood for serum creatinine, PRA, plasma aldosterone, plasma angiotensin II
Collect 24-hour urine for urinary sodium and creatinine

### Visit 6 (Week 4: Day 29)

Study Visit 6 should take place 1 day after Study Visit 5. Patients should be seen for all visits between 7:00 A.M. and 10:00 A.M.

The following procedures are performed:
Assessment of any concomitant medications taken or changed since the last visit.
Assessment of any adverse events that occurred since the previous study visit.
24-hour ABPM is removed
Dispense study medication and complete dosage administration record
Assign sodium level diet.

### Visit 7 (Week 6: Day 42)

Study Visit 7 should take place 2 weeks +/−3 days after Study Visit 6. Patients should be seen for all visits between 7:00 A.M. and 10:00 A.M.

The following procedures are performed:
Blood pressure and pulse rate measurements are performed in the sitting and standing positions. Three blood pressure readings and one pulse rate are taken in the sitting position. After the sitting measurements, one standing blood pressure and pulse rate is taken.
Assessment of any concomitant medications taken or changed since the last visit.
Assessment of any adverse events that occurred since the previous study visit.
Collect 24-hour urine for urinary sodium and creatinine

### Visit 8 (Week 6: Day 56)

Study Visit 8 should take place 2 weeks +/−3 days after Study Visit 7. Patients should be seen for all visits between 7:00 A.M. and 10:00 A.M.

The following procedures are performed:
Blood pressure and pulse rate measurements are performed in the sitting and standing positions. Three blood pressure readings and one pulse rate are taken in the sitting position. After the sitting measurements, one standing blood pressure and pulse rate is taken.
A complete physical exam is performed.
Assessment of any concomitant medications taken or changed since the last visit.
Assessment of any adverse events that occurred since the previous study visit.
Apply 24-hour ABPM
Blood and urine samples are collected in the fasting state for routine safety laboratory evaluations. A serum pregnancy test is performed for women of child bearing potential only.
Collect blood for serum creatinine, PRA, plasma aldosterone, plasma angiotensin II
Collect 24-hour urine for urinary sodium and creatinine

### Visit 9—End of Study Visit (Week 8: Day 57)

Study Visit 9 should take place 1 day after Study Visit 8. Patients should be seen for all visits between 7:00 A.M. and 10:00 A.M.

The following procedures are performed:
Assessment of any concomitant medications taken or changed since the last visit.
Assessment of any adverse events that occurred since the previous study visit.
24-hour ABPM is removed

### Efficacy

Primary efficacy variable is:
Change in mean 24-hour ambulatory systolic blood pressure (MASBP) between diets.

Secondary efficacy variables are:
Change in 24-hour ambulatory mean diastolic blood pressure (MADBP) between diets.
Percentage of responders after each diet compared to baseline (responders are defined as patients with a reduction in MASBP by 20 mmHg or a MASBP of <130 mmHg)

Exploratory efficacy variables are:
1. Change in Plasma Renin Activity (PRA) between diets
2. Change in Angiotensin II between diets
3. Change in Plasma Aldosterone between diets
4. Ambulatory blood pressure during the last 4-hour (hrs 20-24) dosing period between diets
5. Nocturnal and daytime blood pressure response between diets
6. Trough: Peak ratio between diets
7. Smoothness index between diets

### Blood Pressure Measurements

Using a calibrated standard mercury sphygmomanometer and appropriate size cuff, arterial blood pressure determinations are made in accordance with the 1988 AHA Committee Report on Blood Pressure Determination. With the arm supported at the level of the heart, systolic pressure is recorded when the initial sound is heard (Phase I of the Korotkoff sound); diastolic pressure is recorded at the disappearance of the sound (Phase V of the Korotkoff sound). The cuff should be deflated at a rate not greater than 2 mmHg/second.

Sitting and standing blood pressure are measured and recorded at all study visits. At the first study visit, the arm in which the highest sitting systolic blood pressure is found will be the arm used for all subsequent readings throughout the study and should be documented in the source documents.

At each study visit, after the patient has been sitting for about five minutes, with back supported and both feet placed on the floor, systolic and diastolic blood pressures are measured three times using a calibrated standard mercury sphygmomanometer and appropriate size cuff. The repeat sitting measurements are made at approximately 1-2 minute intervals and the mean of these three sitting blood pressure measurements will be used as the average sitting office blood pressure for that visit. The patient then stands and after standing for approximately 2 minutes, one blood pressure measurement is taken.

### Pulse Rate

At each visit, the pulse rate is measured for 30 seconds just prior to the first sitting blood pressure measurement and just prior to the standing blood pressure measurement.

### Ambulatory Blood Pressure Monitoring

24-hour ABPM is conducted three times during the study on all patients. The initial 24-hour ABPM is performed once the patient has qualified for the study based on office cuff blood pressure measurement, but prior to randomization and administration of the first dose of study drug.

The procedure below is followed for each 24-hour APBM period
The ABPM device is attached to the NON-DOMINANT arm of the subject.
A correlation is made between the ABPM device readings and the measurements taken with the sphygmomanometer.

Following the correlation procedure, blood pressure and heart rate is measured using the ABPM device at study specified intervals.

### ABPM Procedures

The following procedures are done over a period of two to three days:

### First Day: Application

The patient appears to the study center, deemed eligible and qualified as per randomization criteria. The patient has all required study procedures completed, including the qualifying office cuff blood pressure. Only patients who meet eligibility criteria undergo ABPM. The ABPM is placed on the non-dominant arm after sphygmomanometer readings between 7:00 am and 10:00 am. Correlation readings are obtained. If the correlation procedure is successful, the coordinator/investigator initiates the “Beginning of Test” reading at Visit 2. The patient is required to return to the clinic within approximately 24 hours to have the ABPM device removed.

Only those patients who fulfill the inclusion/exclusion criteria, complete the Visit 2 ABPM successfully, and have a mean 8-hr. daytime ABPM systolic blood pressure ≧135 mmHg and <160 mmHg will be randomized into the open label treatment period of the study.

### ABPM Removal

The ABPM can be removed after it has been worn for a minimum of 24 hours. The ABPM is downloaded and quality control evaluated on site, using the study specific ABPM software.

At Visit 3, if the criteria for successful readings defined above are not met, ABPM procedures followed on the first day are repeated once, within 24 to 48 hours of the first assessment. The subject is reinstructed regarding ABPM procedures. If the ABPM criterion is met, the coordinator/investigator dispenses the first dose of open label study drug. If a patient does not successfully complete the ABPM after two attempts, the patient is not eligible for the study and will be considered a screen failure.

### Hematology

Hemoglobin, hematocrit, red blood cell count, white blood cell count with differential, and platelet count are measured at Visit 1 and Visit 8.

### Blood Chemistry

Blood Urea, creatinine, plasma glucose, total bilirubin, SGOT, SGPT, LDH, alkaline phosphatase, CPK, sodium, potassium, chloride, calcium, phosphorous, total protein, albumin, bicarbonate and uric acid are measured at Visit 1 and Visit 8. Serum creatinine, PRA and angiotensin II are measured at visits 2, 5 and 8.

Lipid profile (total cholesterol, triglycerides, HDL-C, LDL-C, and VLDL-C) are measured at Visit 1 and Visit 8.

Glycosylated hemoglobin (HbA1c) is performed on all patients with a history of diabetes at Visit 1 only. Patients without a history of diabetes, whose fasting plasma glucose (FPG) level is ≧126 mg/dl at Visit 1, have a reflex HbA1c performed.

### Urine Measurements

Stick-test determination of specific gravity, pH, blood, total protein, bilirubin, ketones, glucose and leukocytes are measured at Visit 1 and Visit 8. If stick is positive, a qualitative microscopic determination (WBCs/HPF, RBCs/HPF) is performed.

24-hour urine will be collected at Visits 2, 4, 5, 7 and 8 and sodium and creatinine is measured.

### Safety Monitoring

### Serious Adverse Event Reporting

To ensure patient safety, every SAE, regardless of suspected causality, occurring after the patient has provided informed consent and until 4 weeks after the patient has stopped study participation must be reported to Novartis within 24 hours of learning of its occurrence.

Any SAEs experienced after this 4-week period should only be reported to Novartis if the investigator suspects a causal relationship to the study drug.

### Data Analysis

### Primary Objective

To assess the change in mean 24-hour ambulatory systolic blood pressure (MASBP) in systolic hypertensive patients treated with aliskiren (300 mg) for 4 weeks on a high sodium diet versus 4 weeks on a low sodium diet.

### Variable

Primary efficacy variable:
Change in mean 24-hour ambulatory systolic blood pressure (MASBP) between diets.

Test for the non-inferiority of the high sodium diet to the low sodium diet in change in mean 24-hour ambulatory systolic blood pressure is based on the following null and alternative hypotheses:

(a) Hₒ: μ_{HS}≧μ_{LS}+4 versus Hₐ: μ_{HS}<μ_{LS}+4,
where μ_{HS} and μ_{LS} are the mean in changes from baseline in 24-hour ambulatory systolic blood pressure for the patients groups with the high and low sodium diet, respectively.

Each test of the high sodium diet group versus the low sodium diet group is a one-sided test at 2.5% significance level.

### Between-Diet Primary Efficacy Analyses

To compare the change from baseline in MASBP between the high and low sodium diet groups, an analysis of variance (ANOVA) model is used. The effects included in the ANOVA model are sequence, subjects within sequence, diet (high sodium, low sodium) and period. Also, a 95% confidence interval for the difference in MASBP between high and low sodium diets groups is reported, based on the fitted model.

### Criteria for Efficacy

The high sodium diet group is considered to be statistically non-inferior to the low sodium diet group in the change in MASBP if the null hypothesis above in (a) is rejected, i.e., if the upper limit of the confidence interval for μ_{HS}-μ_{LS} corresponding to the noninferiority test in (a) does not exceed 4 mmHg (EMEA; 2000).

### Secondary Objectives

To evaluate the change in mean 24-hour ambulatory diastolic blood pressure (MADBP) in systolic hypertensive patients treated with aliskiren (300 mg) for 4 weeks on a high sodium diet versus 4 weeks on a low sodium diet.
To evaluate responder rate defined as a drop in SBP of 20 mmHg or a 24-hour ABPM mean SBP of <130 mmHg from baseline in systolic hypertensive patients treated with aliskiren (300 mg) for 4 weeks on a high sodium diet versus 4 weeks on a low sodium diet.

### Efficacy (Secondary)

Secondary efficacy variables are:
1. Change in mean 24-hour ambulatory diastolic blood pressure (MADBP) between diets.
2. Percentage of responders after each diet compared to baseline (responders are defined as patients with a reduction in MASBP by 20 mmHg or a MASBP of <130 mmHg)

The analyses for the secondary efficacy variable 1 are similar to the primary efficacy analysis.

Secondary efficacy variable 2 is analyzed using a chi-square test with continuity correction appropriate for across-over design. The percent of responders is summarized by frequencies and percentages by week.

### Exploratory Objectives

The exploratory objectives for this trial are the following:
To evaluate Plasma Renin Activity (PRA), plasma angiotensin II and plasma aldosterone in systolic hypertensive patients treated with aliskiren (300 mg) for 4 weeks on a high sodium diet versus 4 weeks on a low sodium diet.
To evaluate other hourly ambulatory blood pressure measurements (i.e. ABPM for the last 4-hour dosing period (hrs 20-24), nocturnal and day time hourly blood pressure (BP) measures, trough to peak ratio and smoothness index) in systolic hypertensive patients treated with aliskiren (300 mg) for 4 weeks on a high sodium diet versus 4 weeks on a low sodium diet.

Exploratory efficacy variables are:
1. Change in Plasma Renin Activity (PRA) between diets
2. Change in Angiotensin II between diets
3. Change in Plasma Aldosterone between diets
4. Mean ambulatory blood pressure measurements during the last 4-hour dosing period (hrs 20-24) between diets
5. Nocturnal and daytime blood pressure response between diets:
   Mean daytime (6 AM-6 PM)
   Mean nighttime (6 PM-6 AM)
   Mean morning (6 AM-12NOON)
6. Trough to Peak ratio between diets
7. Smoothness index between diets

Trough to Peak (TP) ratio is generally defined as a ratio of two BP reductions (trough over peak reduction). Smoothness Index (SI) is the ratio between the mean of the hourly BP reductions and standard deviation

In addition, exploratory analyses are performed separately for “dippers” and “non-dippers”. “Dippers” consists of randomized patients whose mean ABPM nighttime (6 PM-6 AM) systolic blood pressure dropped ≧10% below their mean ABPM daytime (6 AM-6 PM) diastolic blood pressure at baseline. “Non-dippers” consists of randomized patients whose mean ABPM nighttime (6 PM-6 AM) systolic blood pressure did not drop ≧10% below their mean ABPM daytime (6 AM-6 PM) systolic blood pressure at baseline.

For the above exploratory variables, the analyses are performed using the same methods used to analyze the primary efficacy variable. But to compare between the high and low sodium diets, only a two-sided test will be made at the 5% significance level.

1. A method for the prevention of, delay of progression to or treatment of hypertension in a patient with a high sodium diet, comprising administering to such a patient a therapeutically effective amount of a renin inhibitor or a pharmaceutically acceptable salt thereof.

2. A method for the prevention of, delay of progression to or treatment of the effects of a high sodium diet on blood pressure, comprising administering to a patient in need thereof a therapeutically effective amount of a renin inhibitor or a pharmaceutically acceptable salt thereof.

3. The method of claim 1, wherein the renin inhibitor is a compound of formula (I) 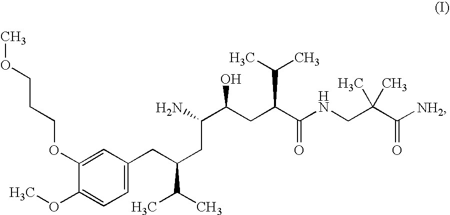
or a pharmaceutically acceptable salt thereof.

4. The method of claim 1 whereby a high sodium diet refers to a sodium intake of ≧200 mmol/day.

5. The method of claim 4 whereby a high sodium diet refers to a sodium intake of 220 to 300 mmol/day.

6. The method of claim 1 whereby the renin inhibitor or a pharmaceutically acceptable salt thereof is used in an amount ranging from 75 to 600 mg of the free base per unit dosage form.

7. The method of claim 1 whereby the blood pressure in said patient can be controlled to be equivalent to the blood pressure of a patient with a low sodium diet undergoing the same treatment.

8. A method of treating hypertension comprising administering
75 to 600 mg of the free base per unit dosage form of a renin inhibitor or a pharmaceutically acceptable salt thereof, is to be administered daily to a patient in need thereof, wherein
the treated patient is selected from a patient has a high sodium diet.

9. The method of claim 8, wherein the renin inhibitor is a compound of formula (I) 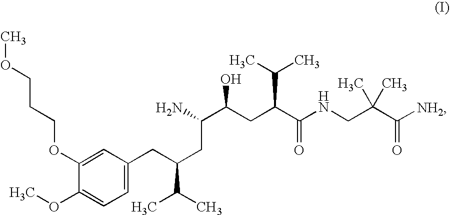
or a pharmaceutically acceptable salt thereof.

10. The method of claim 8 whereby a high sodium diet refers to a sodium intake of ≧200 mmol/day.

11. The method of claim 10 whereby a high sodium diet refers to a sodium intake of 220 to 300 mmol/day.

12. The method of claim 1 whereby the blood pressure in said patient can be controlled to be equivalent to the blood pressure of a patient with a low sodium diet undergoing the same treatment.

13. The method of claim 12, wherein the blood pressure is 24-hour ambulatory systolic blood pressure.
